(19) United States
(12) Patent Application Publication
(10) Pub. No.: US 2021/0069106 A1
(43) Pub. Date: Mar. 11, 2021

(54) HIGH DOSE DELIVERY OF INHALED THERAPEUTICS

(71) Applicant: Novartis AG, Basel (CH)

(72) Inventors: Yoen-Ju SON, Foster City, CA (US); Daniel HUANG, Palo Alto, CA (US); Danforth MILLER, San Carlos, CA (US); Jeffry G. WEERS, Half Moon Bay, CA (US)

(21) Appl. No.: 16/963,678

(22) PCT Filed: Jan. 24, 2019
(86) PCT No.: PCT/IB2019/050607

§ 371 Date: Jul. 21, 2020

Related U.S. Application Data
(60) Provisional application No. 62/622,464, filed on Jan. 26, 2018.

(51) Int. Cl. A61K 9/00; A61K 9/16; C07K 16/24; A61K 31/5383; A61K 31/7036; A61K 31/137; A61M 15/00

(52) U.S. Cl. CPC A61K 9/0075; A61K 9/1617; A61K 9/1682; C07K 16/244; A61M 2202/064; A61K 31/7036; A61K 31/137; A61M 15/0051; A61M 15/0021; A61K 31/5383

(57) ABSTRACT

The present invention comprises methods and formulations to increase drug payload, especially in regard to a receptacle-based, inhalation dosed, dry powder therapeutic, wherein the methods and formulations are characterized by a high product density, as well as a high TLD per receptacle, while maintaining highly efficient aerosol performance from the device. Embodiments of the present invention comprise a spray-dried pharmaceutical powder comprising particles deliverable from a dry powder inhaler, the composition comprising active agent, and a shell-forming excipient, wherein the powder is characterized by a product density greater than 50 mg/ml.

### FIELD OF THE INVENTION

The invention relates to formulations and processes that enable lung delivery of high doses of APIs in a small-volume receptacle, such as a blister or capsule, and to formulations of powders made by such process. Embodiments of the invention comprise dense powders. The powder formulations are useful for the treatment of diseases and conditions, especially respiratory diseases and conditions.

### BACKGROUND

Active pharmaceutical ingredients (APIs) that are useful for treating respiratory diseases are often formulated for inhaled (or pulmonary) administration, such as with portable inhalers. Pulmonary drug delivery methods and compositions that effectively provide the pharmaceutical compound at the specific site of action (the lung) potentially serve to minimize toxic side effects, lower dosing requirements, and decrease therapeutic costs. The development of such systems for pulmonary drug delivery has long been a goal of the pharmaceutical industry.

Inhalation systems and devices commonly used to deliver drugs locally to the pulmonary air passages comprise dry powder inhalers (DPIs), metered dose inhalers (MDIs), and nebulizers. DPIs generally rely entirely on the patient's inspiratory efforts to introduce a medicament in a dry powder form to the lungs. Such dry powder inhalers typically dispense medicaments from receptacles, for example, blisters or capsules. Such receptacles are necessarily limited in volume, typically about 0.1 to 1.5 mL, for example 0.06 to 0.2 mL for blisters and about 0.1 to 1.4 mL for capsules.

Although most asthma and COPD active pharmaceutical ingredients (APIs) are highly potent with lung doses less than 1 mg, a wide range of other APIs, such as antibiotics, are less potent with a required total lung dose (TLD) from a few mg to 10 mg or more. Accordingly, when delivered by typical blister-based or capsule-based inhalation devices, the limited volume of the blister or capsule receptacle often dictates a requirement for multiple inhalation doses in order to achieve therapeutic value.

While a larger receptacle could be used, this would adversely affect the size of the inhaler device, reducing portability, the number of doses contained therein, or both. Receptacle size is therefore an important design constraint for delivery devices, e.g. inhalers, since receptacle size has a bearing on both device design (human factors) and maximum possible therapeutic dose. Receptacle size therefore has a consequence on device form factor, portability and dose administration. It is well-established that patient acceptance, medication adherence, and consequent efficacy are influenced by such human factors engineering. Hence, drug payload, that is, the quantity of drug that can be delivered in a single inhalation, is important to patient-acceptance, adherence and consequent efficacy.

Conventionally, spray-dried respirable particles have been engineered to be of low density, with porous (e.g. PulmoSphere™) or corrugated (e.g. PulmoSol™) surface properties to minimize inter-particulate forces. This maximizes the aerosol dispersibility of the engineered particles—achieving targeted lung delivery while minimizing interparticle cohesive forces. Such particles show improved drug delivery efficiency to the lungs, however the dose range for those engineered particles is narrow due to their low density and poor packing properties. In these approaches, it is important the particle density be kept to a minimum in order to engineer particles within an optimal aerodynamic range.

Several methods have been employed to increase powder fill mass in the receptacles, including increasing the true density of the particles by formulating them with materials with high true density, such as inorganic salts.

In some prior art approaches, workers have attempted formulating with metal cation salts, in an effort to increase the dispersibility of the spray dried powders, thereby enabling a higher dose to be contained within the same unit volume. However these technologies do not achieve fill masses greater than about 40 mg in a size 3 capsule, nor is a calculated product density (as described herein) greater than 40 mg/mL. Formulations employing salts result in only moderate improvements in lung delivery efficiency, and also suffer from a disadvantage in that the metal ion salts can result in hygroscopic formulations which are unstable at high relative humidities.

### SUMMARY OF THE PRESENT INVENTION

Accordingly embodiments of the present invention comprise methods and formulations to increase drug payload, especially in regard to a receptacle-based, inhalation dosed, dry powder therapeutic. Such methods and formulations are characterized by a high product density. “Product density” is a novel metric of the present invention, and governs the total lung dose (TLD) that can be achieved using a device with a fixed volume of receptacles. A high TLD per receptacle can be achieved by increasing the powder fill mass in the fixed volume of receptacles (i.e., product density), while maintaining highly efficient aerosol performance from the device.

Product density is defined by the inventors herein as the mass of drug delivered to the lungs (total lung dose, or TLD) divided by the total volume of the receptacle, and is given by Equation 1:

$${ρ}_{p r o d u c t} = TLD / {V}_{receptacle} = ( \frac{{m}_{p o w d e r}}{{V}_{receptacle}} ) ( \frac{{m}_{d r u g}}{{m}_{p o w d e r}} ) ( \frac{{m}_{lung}}{{m}_{d r u g}} ) \qquad Equation 1$$

For example, for a 150 mg fill mass of a powder with 80% drug loading and a 70% TLD in a size 2 capsule (0.37 mL), the product density would be: (150)*(0.8)*(0.7)/(0.37)=227 mg/ml. In Equation 1, the first bracketed term (powder mass over receptacle volume) relates to the powder filling process, while the second bracketed term (mass of drug over mass of powder) relates to the formulation process, and the final bracketed term relates to drug delivery. “Product density” thus encompasses the amount of powder that is filled into a receptacle, the drug loading in the powder, and the drug delivery efficiency to the lungs. Stated slightly differently, product density is a metric that quantitatively explains collective contributions of multiple aspects or characteristics which influence a lung dose achievable from a given receptacle volume. Such aspects or characteristics include fractional particle density, packing density, inter-particulate forces, and aerosol properties of the particles.

Embodiments of the invention comprise a spray-dried pharmaceutical powder composition comprising particles deliverable from a dry powder inhaler, the composition comprising active agent, and about 0.5 to 10% by weight of a shell-forming excipient, wherein the powder is characterized by a product density greater than 50 mg/ml.

Embodiments of the invention comprise a spray-dried pharmaceutical composition comprising a powder comprising particles made by a process comprising preparing a feedstock comprising a solvent, active agent and 0.5-10% of a shell forming excipient; spray drying the feedstock under process conditions characterized by a Peclet number of 0.5 to 3; and, collecting the resulting powder, wherein the powder is characterized by a product density greater than 50 mg/mL, and a compressibility index of less than 20.

Embodiments of the invention comprise a method of delivering a plurality of particles comprising a therapeutic dose of an active pharmaceutical agent to the lungs of a subject, the method comprising preparing a solution of an active agent and shell-forming excipient in a solvent wherein the shell forming excipient is present between 2 and 5%; spray drying the solution to obtain a powder comprising particulates, wherein the powder is characterized by a product density of at least about 80 mg/mL; packaging the spray-dried powder in a receptacle; and providing an inhaler having a means for extracting the powder from the receptacle, wherein the powder, when administered by inhalation, provides at least 70% lung deposition.

Embodiments of the invention comprise a multiple dose powder inhalation device and drug combination comprising a body comprising an interior cavity and a cartridge that is removably insertable into the interior cavity of the body, the cartridge comprising a mouthpiece through which aerosolized powder medicament may be delivered to a user, wherein the cartridge houses a strip of receptacles, each receptacle adapted to contain a dose of powder medicament, a piercing mechanism to open each blister and an aerosol engine; and a powder medicament contained within each receptacle, wherein the powder medicament comprises a spray-dried pharmaceutical powder composition comprising particles deliverable from a dry powder inhaler, the composition comprising active agent, and about 0.5 to 10% by weight of a shell-forming excipient, wherein the powder is characterized by a product density greater than 50 mg/ml.

Embodiments of the invention provide receptacle-formulation packages having product densities of greater than 60 mg/mL, such as greater than 70 mg/mL, greater than 80 mg/mL greater than 90 mg/mL and greater than 100 mg/mL. Embodiments of the invention provide blister-formulation combinations having product densities of greater than 60 mg/mL. Embodiments of the invention provide capsule-formulation combinations having product densities of greater than 80, 90 or 100 mg/mL.

Embodiments of the present invention comprise methods and formulations to deliver a high drug payload with the device having a small dosing cavity, a minimal number of inhalations, or both. This is especially relevant to a receptacle-based, dry powder therapeutic, dosed via inhalation.

Embodiments of the present invention comprise methods and formulations to increase drug payload without the need to add, or formulate with, salts or other densifying agents, especially in regard to a receptacle-based, dry powder, pulmonarily-dosed therapeutic.

Embodiments of the present invention comprise methods and formulations to design particles that enable creation of a tightly packed powder bed.

Embodiments of the formulation and the process of the present invention result in increased particle density by engineering particles utilizing a spray drying process with a low Peclet number, and wherein surface roughness (rugosity) of particles is controlled to increase the tapped and puck densities of bulk powder.

Embodiments of the present invention provide compositions and manufacturing processes that enable lung delivery of high doses of APIs, having for example a total lung dose requirement of 22 mg or more, in a small-volume receptacle, such as those having a volume of 0.37 mL or less. Embodiments of the present invention provide compositions and processes that enable delivery of conventionally-size doses, in smaller receptacles for example 6 mg or greater in 0.1 mL or smaller volume. Embodiments of the present invention provide compositions and processes that enable delivery of larger-sized total lung doses, in conventionally size receptacles, for example 50 mg or greater in 0.37 mL or smaller volume

In one aspect, embodiments of methods and formulations of the present invention increase the total lung dose (TLD) of an API delivered from a dry powder inhaler. In further aspects, the present invention affords a higher dose to volume ratio, which can in turn lead to smaller and more ergonomically friendly inhalers, and/or multidose inhalers having greater than one month supply of doses, for example two, three, four, five or six month's supply. This enables a multidose inhaler with a one-month (or greater) supply of drug to achieve a total lung dose of up to 10 mg, which in turn enables many new classes of drugs, including, most hormones and antibody fragments, to be delivered in a blister-based multidose dry powder inhaler.

In one aspect, embodiments of methods and formulations of the present invention increase the (TLD) of an API delivered from a small receptacle (e.g. a 0.1 mL blister) inhaler from about 1 mg to more than 6, 7, 8, 9, or 10 mg. In such aspects, the TLD can be 50 to 60% to 70% to 80% to 90% or higher of the receptacle fill mass. This represents a 12 to 20 fold increase in fill mass.

In one aspect, embodiments of methods and formulations of the present invention increase the (TLD) of an API delivered from a medium-sized receptacle (e.g. a 0.37 mL capsule) inhaler from about 19 mg to more than, 50, 100, 150, 200 or 250 mg. In such aspects, the TLD can be 50 to 60% to 70% to 80% to 90% or higher of the receptacle fill mass. This represents a 2.5 to greater than 13 fold increase in fill mass.

In one aspect, embodiments of methods and formulations of the present invention can increase the TLD that can be delivered, via a single inhalation, from a receptacle in a unit dose or single dose disposable dry powder inhaler to more than 100 mg.

In one aspect, embodiments of methods and formulations of the present invention comprise an entire TLD capable of fitting into a single receptacle, and/or being delivered via a single inhalation.

In one aspect, embodiments of methods and formulations of the present invention comprise an entire therapeutic dose capable of fitting into a single receptacle, and/or being delivered via a single inhalation.

In one aspect, embodiments of methods and formulations of the present invention comprise an entire TLD contained within a single receptacle.

In one aspect, embodiments of methods and formulations of the present invention comprise an entire therapeutic dose contained within a single receptacle.

In one aspect, embodiments of methods and formulations of the present reduce the number of handling steps required to administer a therapeutic dose.

Embodiments of the present invention enable a therapeutic dose of tobramycin (TIP®), which is currently administered (via the TOBI Podhaler® inhaler) in four discrete size 2 capsules, to be delivered from two size 2 capsules, or from a single size 2 capsule.

Accordingly, in embodiments of the present invention, there is provided a process to produce a formulation of API which comprises an entire TLD, and/or an entire therapeutic dose, capable of fitting into a single receptacle.

Embodiments of the present invention provide a process for preparing dry powder formulations for inhalation, comprising a formulation of spray-dried particles, the formulation containing at least one active ingredient that is suitable for treating obstructive or inflammatory airways diseases, particularly asthma and/or COPD.

Embodiments of the present invention provide a process for preparing dry powder formulations for inhalation, comprising a formulation of spray-dried particles, the formulation containing at least one active ingredient that is suitable for non-invasively treating diseases in the systemic circulation.

In embodiments of the present invention, the powders are free of added salts or densifying agents.

### Terms

Terms used in the specification have the following meanings:

“Active”, “active ingredient”, “therapeutically active ingredient”, “active agent”, “drug” or “drug substance” as used herein means the active ingredient of a pharmaceutical, also known as an active pharmaceutical ingredient (API).

“Amorphous” as used herein refers to a state in which the material lacks long-range order at the molecular level and, depending upon temperature, may exhibit the physical properties of a solid (glassy supercooled liquid) or a liquid.

“Bulk density” is defined as the ‘apparent’ powder density under different conditions. According to ASTM D5004, the bulk density is the mass of the particles divided by the volume they occupy that includes the space between particles. For the purposes of this invention we measure three bulk densities (i.e., the poured bulk density, the tapped density, and the puck density), that are each determined under specific test conditions.

“Drug Loading” as used herein refers to the percentage of active ingredient(s) on a mass basis in the total mass of the formulation.

“Tapped density” or ρ_{tapped}, as used herein is measured according to Method I, as described in USP <616>. Tapped densities represent an approximation of particle density. Tapped density may be measured by placing the powder material in a sample cell, tapping the material, and adding additional material to the sample cell until it is full and no longer densifies upon further tapping.

“Total lung dose” (TLD) means the percentage of the nominal dose that is deposited in the lungs. In vitro measures of TLD are often determined experimentally with anatomical throat models (e.g., the medium-sized Alberta Idealized Throat) at a pressure drop of 4 kPa. Total lung dose may sometimes be referred to herein simply as “dose”. Dose is to be differentiated from drug “strength”, which is the fill mass multiplied by the drug loading.”

“True Density” is the mass of a particle divided by its volume excluding open pores and closed pores. The true density is often referred to as the pycnometer density, as the true density is typically measured using helium pycnometry.

“Puck density” is the bulk density determined by uniaxial compaction of bulk powder at a pressure of 0.8 bar (24 inHg). The pressure used is representative of that used to compress bulk powder into pucks that are then filled into a receptacle using a drum-based or dosator-based filler.

“Green density” is the mass of the particles divided by the volume they occupy under levels of compression that eliminates free volume to the point that particles are deformed.

“Compressibility Index” (C) is a new metric of the present invention. It provides a measure of the compressibility of a bulk powder, and is given by Equation 2:

C=100(1−ρ_{T}/ρ_{P}), Equation 2

where ρ_{T} is the tapped density and ρ_{P} is the puck density. This differs from Carr's Index, which utilizes the poured bulk density and tapped density. The compressibility index as described herein is a better correlate for powders filled on drum fillers which create powder pucks using powder compression.

“Delivered Dose” or “DD” as used herein refers to an indication of the delivery of dry powder from an inhaler device after an actuation or dispersion event from a powder receptacle. DD is defined as the ratio of the dose delivered by an inhaler device to the nominal or metered dose. The DD is an experimentally determined parameter, and may be determined using an in vitro device set up which mimics patient dosing. DD is also sometimes referred to as the emitted dose (ED).

“Median aerodynamic diameter” (MAD) of the primary particles or Dₐ as used herein, is calculated from the mass median diameter of the bulk powder as determined via laser diffraction (x50) at a dispersing pressure sufficient to create primary particles (e.g., 4 bar), and their tapped density, that is: Dₐ=x50 (p_{tapped})^{1/2}.

“Primary particles” refer to the individual particles that are present in an agglomerated bulk powder. The primary particle size distribution is determined via dispersion of the bulk powder at high pressure and measurement of the primary particle size distribution via laser diffraction. A plot of size as a function of increasing dispersion pressure is made until a constant size is achieved. The particle size distribution measured at this pressure represents that of the primary particles.

Throughout this specification and in the claims that follow, unless the context requires otherwise, the word “comprise”, or variations such as “comprises” or “comprising”, should be understood to imply the inclusion of a stated integer or step or group of integers or steps but not the exclusion of any other integer or step or group of integers or steps.

The entire disclosure of each United States patent and international patent application mentioned in this patent specification is fully incorporated by reference herein for all purposes.

### DESCRIPTION OF THE DRAWINGS

The formulations, compositions and methods of the present invention may be described with reference to the accompanying drawings. In those drawings:

FIG. 1 is a schematic illustration of droplet drying, showing morphological changes over time.

FIG. 2 is a graph a particle size and density versus Peclet (Pₑ) number of a trileucine aqueous system. Particle size is shown by the curve labeled with squares and beginning lowest on the Y axis. Density is reported by the curve labeled with diamonds.

FIG. 3 is a diagrammatic illustration of various types of densities and a coordination number (Nc) associated therewith. For purposes herein, the coordination number represents the number of particles touching a given particle, and increases with powder densification.

FIG. 4 is a scanning electron photomicrograph of a fine non-engineered spray dried powder without shell-forming agent, made in accordance with Example 7 (Table 2—lot 761-58-10) showing the void spaces (bound regions in the photomicrograph) which result in undesirably low tapped densities.

FIG. 5A is a graph of Compressibility index, and FIG. 5B is a graph of Carr's index. FIG. 5 show the emitted dose of spray-dried powders comprising an antibody fragment, expressed as a function of percentage fill mass versus Carr's index (5B) and Compressibility index (5A). A target fill mass of 150 mg of powder was filled into HPMC capsules for the emitted dose testing.

FIGS. 6A, 6B, and 6C are scanning electron photomicrographs of spray dried particles comprising an antibody fragment. FIG. 6A shows particles made in accordance with Table 2 Example 7 (0% shell former). FIG. 6B shows particles made from a formulation comprising antibody fragment and leucine (not shown in Table 2). FIG. 6A thus shows particles with 0% shell-former (characterized by a smooth particle morphology) and which were spray dried at fast drying conditions—a low Pe. FIG. 6B shows particles with 10% shell-former (as leucine), also spray dried, but under spray drying conditions resulting in a higher Pe relative to that for the particles shown in FIG. 6A. It can be seen that, in part, owing to the presence of the shell former, the particles in FIG. 6B exhibit a dimpled morphology. FIG. 6C shows particles with 15% shell-former (as trileucine) spray dried under fast drying conditions (high Pe) exhibiting an undesirable (for high payload applications) corrugated morphology.

FIG. 7A is a graph of specific surface area (SSA) of a spray-dried powder comprising antibody fragment versus shell-former content (as trileucine), showing that the SSA increases with trileucine content. Points comprising formulations produced under fast drying conditions (high Pe) are plotted as diamonds and points comprising slow drying conditions (low Pe) are plotted as squares. FIG. 7B is a graph of emitted dose expressed as percent of fill mass versus surface area (all for the same powder formulation) showing that powders manufactured with a low Pe have a lower SSA, higher emitted dose values and less variability in emitted dose, considering the high (greater than 100 mg) fill mass. The dashed-line box in both figures delimits embodiments of powders dried at low Pe, and which exhibit desired performance. The powder analyzed for FIGS. 7A and 7B comprises an antibody fragment made in accordance with Example 2.

FIG. 8 is a graph of bulk and tapped density versus trileucine content of a spray dried powder comprising antibody fragment, showing the impact of increasing shell forming excipient on bulk and tapped density of spray dried formulations in accordance with Example 2.

FIG. 9 is a graph of nominal drug mass (in mg) versus receptacle volume (milliliters) and wherein four different curves are plotted at 70% total lung delivery. Curve A (represented by a dotted line) is product density at 40 mg/mL. curve B (represented by a spaced dotted line) represents product density at 60 mg/mL Curve C (the dashed-dotted line) is product density at 80 mg/mL and curve D (the dashed line) represents product density at 100 mg/mL. Three product density data points are also plotted on the graph, one representing an embodiment of the commercial PilmoSphere formulation of tobramycin inhalation powder (TIP), a second representing an antibody fragment (Fab) and the third representing a formulation of levofloxacin. The Fab and levofloxacin formulations are spray dried powder formulations according to embodiments of the present invention and made in accordance with examples in Table 5.

### DETAILED DESCRIPTION

Embodiments of the present invention are directed to a process and powder formulations which formulations are characterized by high total lung dose for a given receptacle volume. In embodiments of the invention, high total lung doses of APIs capable of being contained within a small-volume receptacle, such as a blister or capsule.

In embodiments of the present invention, the formulations herein are characterized by a high ‘product density’, which is a function of several important aspects of high dose delivery. Product density is defined specifically in Equation 1, which incorporates a term for the powder filling process, a term for the powder formulation process, and a term for the powder delivery system. The product density is defined as the total lung dose (TLD) of the API (mg) divided by the volume of the receptacle within which the dose is contained (mL). Hence such characteristics (e.g., formulation, powder manufacturing, filling, packaging, and aerosol performance, are important aspects to define product density and therefore the present invention. Purely for illustrative purposes, Table 1 below shows standardized capsule sizes and their corresponding capacity in milliliters.

TABLE 1

| Size | Volume (mL) |
|---|---|
| 000 | 1.37 |
| 00 | 0.95 |
| 0 | 0.68 |
| 1 | 0.50 |
| 2 | 0.37 |
| 3 | 0.30 |
| 4 | 0.21 |
| 5 | 0.13 |

The receptacle can be a blister, capsule, pod or other unit volume container. In some embodiments, a receptacle volume may be about 0.37 ml or less (i.e., a size 2 capsule). It has been determined that most patients can empty the contents of powder from a size 2 capsule in a single inhalation. In some embodiments, a receptacle volume may be about 0.30 mL or less (i.e., a size 3 capsule). In some embodiments, a receptacle volume may be about 0.50 mL or less (i.e., a size 1 capsule). In some embodiments, the receptacle volume may be about 0.1 mL or less, for example a blister.

The TLD can be obtained using an anatomical throat model (e.g., the Alberta Idealized Throat, AIT model). TLD is dependent on the drug loading in the formulation, the powder fill mass, and the aerosol performance of the formulation when delivered with a portable dry powder inhaler.

Embodiments of formulations of the present invention comprise product densities that are greater than 60 mg/ml, such as greater that 70 or 80 or 90 or 100 mg/ml. Where powder is filled into capsules, product densities may be even higher, such as greater than 200 mg/ml or greater than 250 mg/ml. This is up to 6-fold higher than the best product densities currently achieved, for example for tobramycin inhalation powder, currently marketed by Novartis as TOBI® Podhaler® TIP, the product achieves a product density of about 48 mg/mL. In embodiments of the present invention, a product density is between 60 mg/mL to 300 mg/m, as well as any value or range of values between.

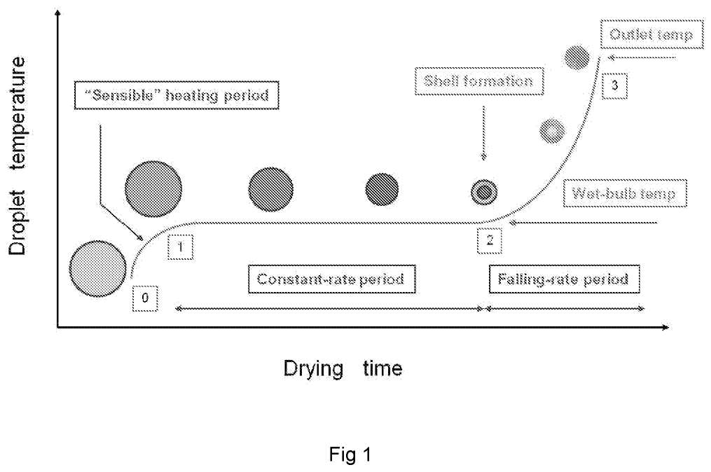

Such desirably high product densities are obtained by embodiments comprising suitable particle engineering of inhaled therapeutic formulations. Spray drying is a suitable technology to obtain engineered particles. FIG. 1 is a graph of droplet and particle temperature as a function of drying time, and shows schematically morphological changes that occur in the droplet over time. As can be seen from FIG. 1, during the sensible heating period (that is the heating period which exhibits temperature increase versus latent heat), the droplet temperature increases to its wet-bulb temperature. During the constant-rate drying period, the droplet behaves like pure solvent; the evaporation rate is dictated by wet-bulb drying kinetics. At the wet-bulb temperature, the droplet shrinks as the solvent is rapidly lost through evaporation. As evaporation progresses, solute molecules (or emulsion droplets, or suspended particles) arrange themselves within the droplet according to diffusion rates. When solidification occurs (also called skin formation), it is the beginning of falling-rate drying period. During this stage, further shrinkage can occur, and the skin may collapse or fracture depending on the material properties. The skin temperature increases as liquid boundary moves inward. At this point, solidification slows the transport of solvent to the surface for evaporation and drying becomes diffusion rate-limited. It has been recognized that particle formation during droplet drying is the most important process controlling the size, density, composition distribution, and morphology of spray-dried particles. Both experimental data and theoretical analysis have demonstrated that the interplay of the rates of solvent evaporation and solute diffusion during the constant-rate period of drying process results in the formation of particle with specific characteristics. Therefore, the Peclet number is used herein to provide insight into the particle formation mechanism during spray drying.

One of the important parameters controlling spray-dried particles with target properties is the Peclet number (Pe), a dimensionless number which is connected to heat and mass transfer rates in the transport phenomena. Peclet number is defined as the ratio of the rate of materials transported by thermal energy to the rate of materials transported by concentration gradient. Stated another way, Pe is the ratio of liquid evaporation to solute diffusion In a spray drying process. The Pe is concerned with the interface of evaporation of solvent and solute accumulation of an individual droplet during constant-rate of drying period. As a result, the Pe can be defined by Equation 3:

$$Pe = \frac{evaporation rate}{diffusion rate} = \frac{k}{D} \qquad Equation 3$$

where k is the solvent evaporation rate and D is the solute diffusivity. For clarity, Peclet numbers referred to herein in conjunction with a powder referred to that aspect of the production process, and not to the powder itself.

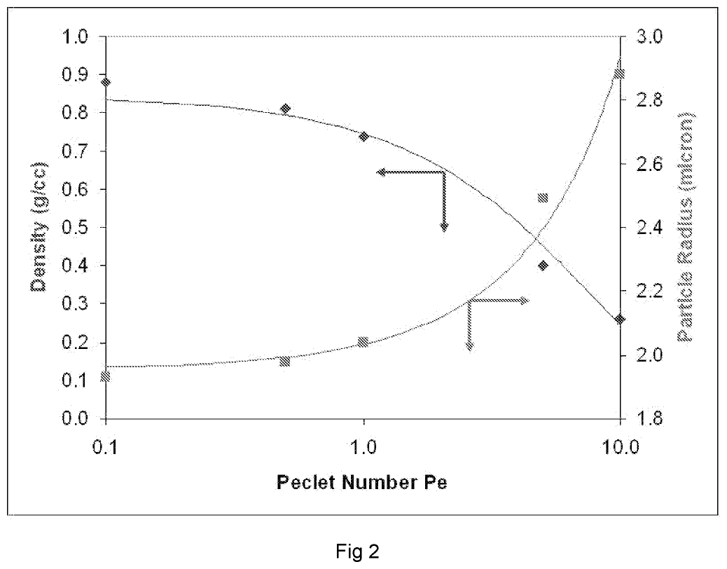

FIG. 2 illustrates how particle size and density are influenced by Pe. In general, at low Pe, both particle size and density change gradually since the rate of solvent evaporation is slower than that of solute diffusion. This allows sufficient time for solute molecules to diffuse toward the center of the droplet resulting in formation of a small solid particle. Under this circumstance, particles form a dense structure close to the theoretical density of the material. As Pe increases, solute enrichment on the surface of atomized droplets is accelerated since the solute molecules in the media do not have enough time to diffuse and distribute within the droplet. The faster the evaporation rate, the sooner the surface reaches its critical supersaturation, causing early skin formation. This condition will lead to a larger particle size and a lower-density with a wrinkled and/or hollow particle morphology.

Embodiments of the present invention comprise fine spray-dried particles (having a primary particle size, X50=1-3 μm) with Pe from about 0.5 to 3, such as between 0.7 to 2, that comprise small amounts of a shell-forming excipient, such that the specific surface area of the particles in the presence of the shell-forming excipient is comparable to like-sized particles comprising no shell-former. That is to say, that the specific surface area is not significantly altered by the presence of such small amounts of shell forming excipient (see also FIG. 7A). Specific surface area depends on the size of the particles and upon the surface morphology. This means that the impact of increasing particle rugosity can be masked if the comparator particle is of a smaller size with a greater specific surface area. Hence comparisons of specific surface area are made by comparing to a smooth particle of the same size.

It is thought the shell-forming excipient reduces interparticle cohesive forces which govern the packing density of the particles as well as the aerosol performance. Lower cohesive forces reduce the void volume between particles in the bulk powder, enabling significant increases in tapped density and puck density and, in turn, product density. Further, reduced cohesive forces lead to improved powder fluidization and dispersion even when the powder is compressed. This is achieved without resorting to use of a metal ion salt to increase the true density of the materials and resulting particle density.

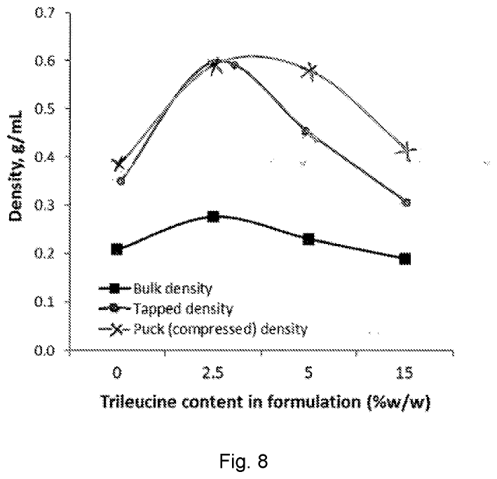

Embodiments of the present invention result in a powder comprising particles with superior packing properties (higher tapped density). This is achieved by particle engineering to achieve a specifically designed fractional density and by controlling inter-particulate forces of particles. If particles are too corrugated, inter-particulate forces will be minimized but the fill mass will be significantly lower due to low particle density. On the other hand, if particles are too smooth and spherical, the fill mass will also be low due to the void spaces created by particle ‘bridging’ (that is, a form of particle agglomeration) in the powder bed. FIG. 8 shows an example of the dependence of powder packing (tapped density) on shell-former (in this case—trileucine) content. Because trileucine induces surface roughness, the x axis can be considered an indirect measure of surface roughness. The shapes of the curves show a maximum at a trileucine content intermediate to extremes of no trileucine and 15% w/w trileucine. Thus, between these two extremes, there is a desired particle morphology to optimize packing properties. Accordingly, in embodiments of the present invention it has been found that powder packing can be significantly improved by introducing a small amount of a shell-forming agent (for example, 2.5-5% w/w of trileucine). This introduces surface roughness to minimize inter-particulate forces of the particles in the bed. Since the amount of shell-former added to the formulation was minimal, the fractional density of the particles was not significantly lowered when compared to particles with highly corrugated surfaces.

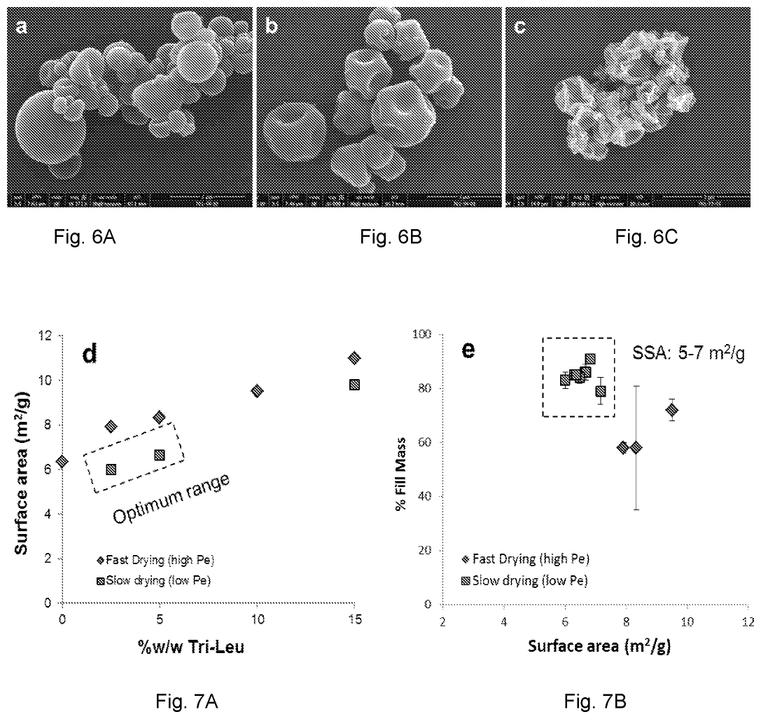

It was also found that densifying the powder by significantly reducing particle rugosity is not an ideal way to increase fill mass and total lung dose. As shown in the SEM image of FIG. 6A, particles formulated without a shell-forming agent (no leucine or tri-leucine) have a smooth, spherical shape. Theoretically, smooth spherical particles have a higher fractional density than corrugated particles, and would therefore pack more efficiently in a fixed-volume receptacle. However, the packing of smooth spherical particles is not only a function of their fractional density, but also greatly influenced by the size of the particles. Spherical particles, larger than about 100 μm have the best packing density due to their weak inter-particulate force. Such particles are gravitationally stable, indicating they can be packed by gravitational forces, which greatly exceed cohesive forces. Contrary to the case for large spherical particles, small spherical particles (less than about 100 μm) are gravitationally unstable; that is, cohesive forces have a greater influence on particle packing than the gravitational forces. FIG. 6B shows particles of increasing rugosity, as dictated by slow drying conditions and 10% leucine in the formulation. FIG. 6C shows the undesirable corrugations arising from fast drying conditions and 15% tri-leucine.

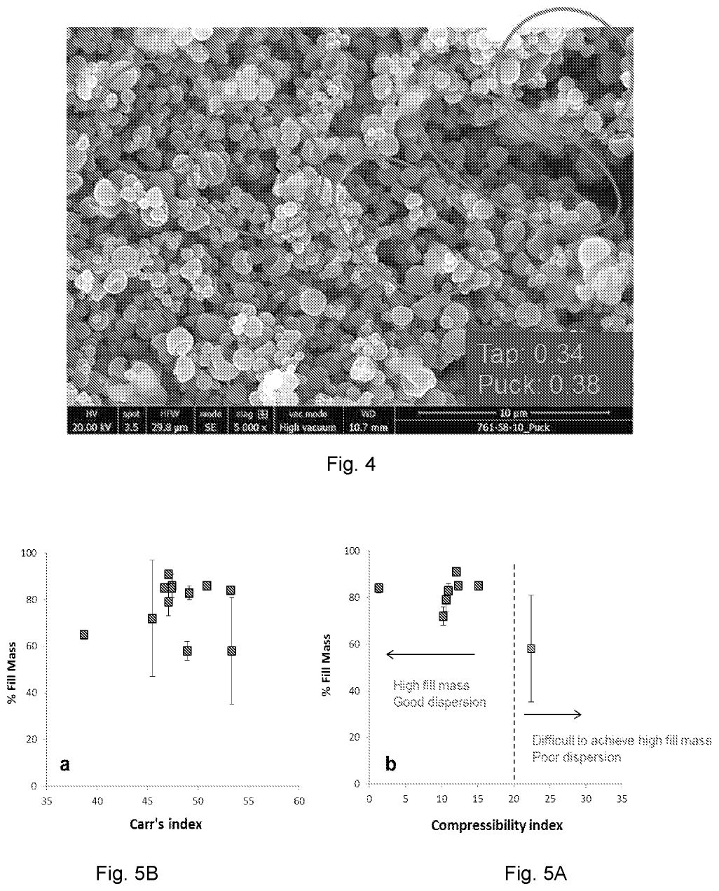

FIG. 4 is an image of a powder bed created by compressing smooth spherical particles (X50: 1.2 μm). The formulation was as shown in Table 2, Example 7. The powder shown in FIG. 4 exhibits a tapped density of 0.34 g/cm³ and puck density of 0.38 g/cm³. Even if individual particles have a smooth spherical morphology providing for a high fractional density, tight packing could not be achieved due to the large agglomerates formed which create large void spaces in the powder bed. As summarized in Table 2, the puck density and fill mass of the smooth particles were lower than those for the corrugated particles due to the strong inter-particulate forces, which is also in good agreement with the particle packing observed in SEM images. Besides the low fill mass, the aerosol performance of formulation A was expected to be poor due to the strong inter-particulate forces.

Embodiments of the present invention provide particles with low rugosity that are gravitationally stable with weak interparticle cohesive forces. In embodiments of the invention, such particles are generally spherical. The Compressibility Index (C) was found by the inventors herein to correlate with dispersibility properties of the powders as filled in the receptacle. In a dispersible powder, ρ_{Tapped} and ρ_{Puck} are close in value and C is small (i.e., less than about 15%). For C greater than about 20, dispersibility is decreased. The Compressibility Index is similar to Carr's Index which utilizes the bulk density and tapped density. It was found that the Compressibility Index correlates better with aerosol dispersibility properties of the formulation than does Carr's index. While Carr's index is conventionally used to predict powder flow, it is relevant only for powders having a relatively large (geometric/aerodynamic) diameter. Therefore, it is not particularly meaningful when considering characteristics of engineered inhalation powders. Such powders generally flow poorly compared to other forms of pharmaceutical powders.

In embodiments of the present invention, there is an optimum rugosity, an optimum compressibility Index and optimum spray drying conditions (the latter characterized by Peclet number). Generally, higher values of each result in lower density because either or both the particles themselves are low density, or the particles form agglomerates due to cohesive interparticle forces. Lower values result in higher density because the particles are more corrugated.

FIG. 5A is a plot of Compressibility Index (“CI”) versus percent fill mass for various formulations. It can be seen that formulations with a Compressibility Index below about 20 enable both a high fill mass and good dispersibility (ED greater than 70, such as greater than about 80). A formulation with a Compressibility Index of higher than about 20 (shown by the data point to the right of the vertical dashed line) exhibited poor dispersibility. FIG. 5B is a plot of emitted dose (expressed as a percentage of fill mass) versus Carr's Index. These two graphs show that the compressibility index correlates better with aerosol dispersibility then does Carr's index. Expressing compressibility in terms of the tapped density and puck density better aligns with the bulk densities which is relevant to the manufacturing process (e.g., in machine filling of receptacles).

As shown by the point to the right of the CI=20 line in FIG. 5A, filling of high masses of the conventional corrugated (e.g. Pulmosol) powders into a fixed volume of receptacle is difficult to achieve. Only one formulation: (Table 2, Example 9) among all the formulations prepared with a conventional Pulmosol formulation process reached the target 150 mg fill mass. See Table 2, Examples 7-12. However, the aerosol performance of this conventional Pulmosol formulation, particularly in the therapeutic-relevant emitted dose criterion, was low and variable, hence unsuitable for use. It is believed this is because the powder required tight compression in order to achieve the target fill mass. In contrast to the conventional formulations, formulations of the present invention readily reach the target 150 mg fill mass without the need for substantial compression, and while maintaining superior aerosol performance, as shown, for example by an emitted dose of greater than 80 to 90%. See Table 2 Examples 1-5 and FIG. 5A (points to the left of the CI=20 line).

In embodiments of the present invention, it has been discovered that a low Compressibility Index can be obtained for fine particles, for example, those in the size range from 1 to 5 μm. Importantly, powders with a low Compressibility Index exhibit improved powder fluidization and dispersibility following compression. This discovery is surprising in view of the prior art in that comparative engineered formulations tend to have high compressibility (for example greater than about 20) because they exhibit a degree of corrugation and a low density.

### Formulation/Particle Engineering

Embodiments of the invention comprise methods and materials for preparing high doses of APIs in a small-volume receptacle, such as a blister or capsule, and to formulations of powders made by such process

Embodiments of the invention comprise a process whereby an API can be formulated to have a product density 1.5 to 7 times greater than that of a conventional inhalation powder, such as 2-6 times greater or 3-5 times greater.

Embodiments of the invention comprise a process whereby an API can be formulated to have a product density of 50 or 100 or 150 or 200 or 250 or 300 or 350 or 400 or 500 or 600 or 700% greater than that of a conventional low-density engineered powder.

In embodiments of the present invention, the formulation is designed to accomplish at least one or more of (a) maximize drug loading by minimizing excipients and/or high molecular weight counter ions; (b) maximize the true density of the components making up the particle without negatively impacting chemical and physical stability of the drug product; (c) maximize particle density (i.e., to minimize void structures or pores within particles); (d) maximize the bulk density of the powder (i.e., to minimize free volume between particles), and; (e) maximize aerosol delivery efficiency to the lungs. Hence, in embodiments of the present invention one, two, three, four, or five of these features are utilized to maximize product density as defined herein. Additionally, by minimizing free volume in a receptacle, such as a capsule, as a part of the filling process, product density may be further increased.

Particle density can be maximized in at least two ways: (1) by formulating with excipients with a high true density (e.g., metal ion salts), and (2) by creating particles under low Pe conditions. From particle engineering considerations, Pe depends both on the formulation composition as well as the process conditions. For particles produced with a low Pe (i.e., 0.5<Pe<3), there is sufficient time for solutes to diffuse throughout the evaporating droplet. Such formulations comprise solid particles with a small geometric size, and a particle density closer to the true density of the components. Experimentally determining particle density can be difficult. Often, the tapped density is used as a surrogate for particle density. However, the tapped density also contains contributions from the free (interstitial) spaces between particles, hence underestimates particle density. This interstitial space can be quite large, especially in ensembles of cohesive particles.

In contrast, particles made by a process using a high Pe (i.e., a Pe between about 3 and 10), comprise low density core-shell particles. Generally speaking, at a very low Pe the particle will be spatially homogenous. At a very high Pe complete phase separation will occur, resulting in a “pure” core-shell particle. At intermediate values of Pe there will be a concentration gradient in the dried particle. For formulations comprising a shell-forming excipient (e.g., leucine or trileucine), the core-shell particles may comprise corrugated particles whose surface is enriched in the shell-forming excipient, and a core comprising the drug substance and other excipients (e.g., buffers, glass-forming excipients, antioxidants, etc.) needed to physically and chemically stabilize the API.

A corrugated morphology reduces cohesive forces between particles, enabling formulations with improved lung targeting (i.e., high lung delivery efficiencies and decreased off-target delivery). This improves dose consistency relative to formulations comprising lactose blends or spheronized particles. While low density core-shell particles alone are suboptimal for maximizing product density, it has been surprisingly discovered that the use of small amounts of a shell-forming excipient to induce some surface corrugation in largely solid, smooth, finely-sized particles with low Pe, beneficially decreases interparticle cohesive forces. This not only enables improvements in aerosol performance, but is also important in maximizing product density. Reducing cohesive forces plays a significant role in increasing the tapped density of the spray-dried powder. These powders were found to have a low Compressibility Index, consistent with limited free volume in the bulk powder. For particles with a high Compressibility Index (e.g., low-density, corrugated particles with a high Pe), increases in fill mass by compression of the powder significantly reduces the ability to effectively fluidize the powder and, in turn, decreases the emitted dose when delivered with a portable dry powder inhaler. In contrast, for powders with a low Compressibility Index, compression of powder has less influence on emitted dose.

Low Pe particles are typically smaller in size, which enables high efficiency lung delivery only if a small percentage of shell former is present to reduce interparticle cohesive forces to improve powder fluidization and dispersion. To achieve efficient lung delivery, the geometric size of the particles should be less than 5 μm, more typically between 1 μm and 3 μm. The Pe for a given formulation depends on the composition of the feedstock to be spray dried and the process parameters. To put it simply, the goal is to decrease the concentration of the shell former, and to dry the particles slowly, thereby allowing time for solutes within the particle to diffuse more uniformly throughout the evaporating droplet, which leads to formation of particles with mild corrugation or dimpling on the surface.

To this end, the concentration of shell former depends on the physical properties of the shell former and percent saturation (i.e., ratio of shell-former concentration to its equilibrium solubility) in the feedstock. In general, it is desired that the ratio of the shell-former to its equilibrium solubility be greater than the ratio of drug and any other dissolved solutes to their equilibrium solubilities. This ensures that the shell-former precipitates first during evaporation. That is, it is important to ensure that the correct component, that is the shell former, forms the outside of the particle.

In embodiments of the invention comprising leucine, e.g. mono- di- or tri-leucine as the shell former, the optimal concentration in the solid particles is less than about 5% w/w, such as less than 4% or 3% or 2.5% w/w. A practical minimum amount of leucine is 0.5%. Therefore, embodiments of the present invention may utilize trileucine in any value between about 0.5% and 10%. Owing to its greater solubility in water, the optimal loading of leucine is expected to be higher than is observed for trileucine, and can be determined without extensive experimentation. Appropriate weight percentages of other oligomers of leucine can be readily determined considering their percent saturations. As a practical matter, the concentration of shell former should be such that a desirably low Pe (less than about 3) results from the process, as well as significantly higher bulk densities (i.e., both tapped densities and puck densities). Despite the significant increase in bulk density, the specific surface area (SSA) of the particles is comparable to that achieved in the absence of a shell-forming excipient, suggesting that the packing of the particles (bulk and tapped density) is improved while maintaining their particle density. Importantly, the tapped density has been significantly increased to values greater than 0.5 g/ml.

Other shell-formers may be utilized, and may provide the desired benefits at concentrations below 0.5% and/or concentrations above 10%.

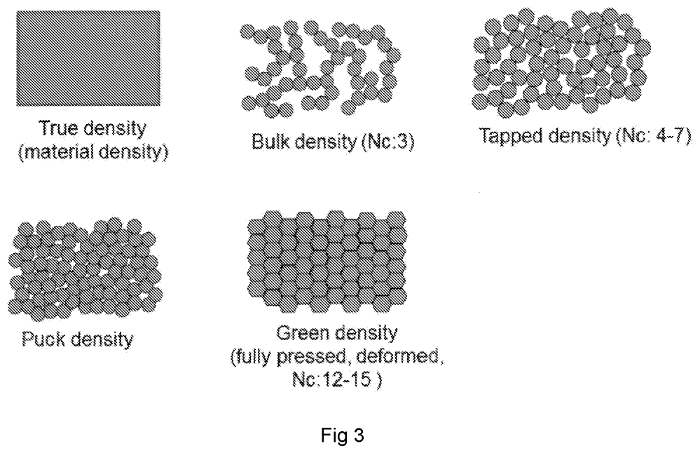

For particles larger than 100 μm, gravitational forces exceed interparticle cohesive forces. Under this scenario, spherical particles have the most efficient packing density. For such large particles, the bulk density decreases with increasing rugosity (measure of small-scale variations of amplitude in the height of a surface). However, as the particle size decreases to less than 10 μm, interparticle cohesive forces exceed gravitational forces and particle morphology takes on greater importance. In this case, smooth, spherical particles may have a lower coordination number and decreased bulk densities. Referring again to FIG. 3, various material densities are illustrated schematically and associated with a coordination number (Nc). The coordination number represents the number of touching neighbors for each particle, and increases with powder densification. Cohesive smooth spherical particles create large void spaces between agglomerates, shown by the circled portions of FIG. 4, thus these ensembles of particles have a low tapped density. However, it should be noted that a low tapped density does not necessarily mean that the particles themselves are of low density. Hence, some degree of particle rugosity is important for ‘respirable’ sized particles to reduce interparticle cohesive forces and increase coordination number.

“Rugosity” as used herein is a measure of the surface roughness of an engineered particle. For the purposes of this invention, rugosity is calculated from the specific surface area obtained from BET measurements, true density obtained from helium pycnometry, and the surface to volume ratio obtained by laser diffraction (Sympatec), Rugosity=(SSA·ρ true)/Sv where Sv=6/D32, where D32 is the average diameter based on unit surface area. Increases in surface roughness are expected to reduce interparticle cohesive forces, and improve targeting of aerosol to the lungs. Improved lung targeting is expected to reduce interpatient variability, and levels of drug in the oropharynx and systemic circulation.

For example, in embodiments of the present invention a particle rugosity may be between about 1 and 3.5, such as 1 to 3, or 1.5 to 2.5

Embodiments of the present invention comprise spherical particles with low rugosity that are gravitationally stable with weak interparticle cohesive forces.

The Compressibility Index (C) correlates with dispersibility properties of the powders filled into receptacles. In a free-flowing powder, tapped density (ρ_{Tapped}) and puck density (ρ_{Puck}) are close in value and C is small (i.e., less than about 15). For C greater than 20, dispersibility is decreased.

Formulation composition and spray-drying process parameters both influence particle morphology. In practice, after inter-particulate forces are minimized by adjusting the formulation composition, the packing density (tapped density) can be further increased by adjusting the spray-drying process parameters. As summarized in Table 2, the tapped density of the particles with the same formulation composition varied with the drying conditions; particles spray dried under slow conditions (sample 4) packed better that the one dried under fast conditions (sample 8). This result indicates that the particles dried under mild conditions (low Pe) have higher fractional density due to lower surface roughness than the one dried under fast conditions (high Pe). The specific surface area (SSA) results (Table 2) are consistent with the tapped density results. Table 2 also shows that formulations dried under fast conditions (samples 7 through 12) showed either poor aerosol properties or poor powder packing (most formulations did not reach the target fill mass, 150 mg in a size 2 capsule).

### Process

During the spray drying process, the bulk feedstock is atomized to a flume of droplets using a nozzle. Control of a droplet size distribution is essential for the consistent and efficient production of spray dried particles for inhalation drug delivery. The final product particle size can be estimated, by equating the mass of dissolved solids to the mass of the dried particle yielding the following Equation 4:

$${d}_{particle} = \sqrt[3]{( \frac{Cs \cdot {ρ}_{solution}}{{ρ}_{particle}} )} \cdot {d}_{droplet} \qquad Equation 4$$

where d_{particle} is the particle diameter; d_{dropiet} is the droplet diameter; Cₛ is the solution concentration or total solids; ρ_{particle} is the particle density; and ρₛₒₗᵤₜᵢₒₙ is the solution density. Hence the final product particle size is controlled predominantly by the initial liquid droplet size and solution concentration.

In some embodiments, a twin-fluid atomizer is employed, which utilizes a high-speed gas stream, typically air, to blast the liquid into droplets. The atomization is achieved by using the kinetic energy of the gas stream provided by a compressed source with typical pressures operating up to 100 psi. The nature of the feedstock is important in achieving low Pe. The solids content should be low enough to prolong the constant-drying period, thus delaying the time to reach supersaturation, where skin formation would occur to achieve a low Pe. In other words the lower the Pe that is attained during the process, the smaller, and more dense is the resulting powder.

To maximize a delivered dose, in addition to increasing product density, it is also important to fill as much of the volume in the receptacle as is possible without negatively affecting powder fluidization and dispersion during inhalation with a dry powder inhaler. The mass of drug that can be loaded into a receptacle depends on the free volume present in the particle (i.e., its porosity), the free volume between particles in the compressed powder puck, and the free volume in the receptacle not occupied by the powder puck. The first two free volumes are assessed in the measurement of the puck density.

Drum or dosator-based fillers that are typically used for the filling of spray-dried powders create a nearly cylindrical puck of powder in predefined shapes, for example, a truncated cone. When pucks are placed in the receptacle, significant free volume is typically observed. Careful design of the puck size and shape may enable a greater percentage of the receptacle volume to be filled, particularly if multiple pucks are filled into the receptacle. Alternatively, the powder may be compressed within a receptacle and additional pucks added subsequently. Other powder filling strategies may be applicable as known to the art, consistent with the teachings herein.

Spray-drying comprises four unit operations: feedstock preparation, atomization of the feedstock to produce micron-sized droplets, drying of the droplets in a hot gas, and collection of the dried particles with a bag-house or cyclone separator. Embodiments of the spray drying process of the present invention comprise the latter three steps, however in some embodiments two or even all three of these steps can be carried out substantially simultaneously, so in practice the process can in fact be considered as a single-step unit operation.

In embodiments of the present invention, a process of the present invention which yields dry powder particles comprises preparing a solution feedstock and removing solvent from the feedstock, such as by spray drying, to provide the active dry powder particles.

In embodiments of the invention, the feedstock comprises at least one active dissolved in an aqueous-based liquid feedstock. In some embodiments, the feedstock comprises at least one active agent dissolved in an aqueous-based solvent or co-solvent system. In some embodiments, the feedstock comprises at least one active agent suspended or dispersed in a solvent or co-solvent system.

The particle formation process is complex and dependent on the coupled interplay between process variables such as initial droplet size, feedstock concentration and evaporation rate, along with the formulation physicochemical properties such as solubility, surface tension, viscosity, and the solid mechanical properties of the forming particle shell.

In some embodiments the feedstock is atomized with a twin-fluid nozzle, such as that described in U.S. Pat. Nos. 8,936,813 and 8,524,279. Significant broadening of the particle size distribution of the liquid droplets can occurs above solids loadings of about 1.5% w/w.

In some embodiments, narrow droplet size distributions can be achieved with plane-film atomizers as disclosed for example in U.S. Pat. Nos. 7,967,221 and 8,616,464, especially at higher solids loadings. In some embodiments, the feedstock is atomized at solids loading between 0.1% and 10% w/w, such as 1% and 5% w/w.

Any spray-drying step and/or all of the spray-drying steps may be carried out using conventional equipment used to prepare spray-dried particles for use in pharmaceuticals that are administered by inhalation. Commercially available spray-dryers include those manufactured by Büchi Ltd. and Niro Corp.

In some embodiments, the feedstock is sprayed into a current of warm filtered air that evaporates the solvent and conveys the dried product to a collector. The spent air is then exhausted with the solvent. Operating conditions of the spray dryer such as inlet and outlet temperature, feed rate, atomization pressure, flow rate of the drying air, and nozzle configuration can be adjusted in order to produce the required particle size, moisture content, and production yield of the resulting dry particles. The selection of appropriate apparatus and processing conditions are within the purview of a skilled artisan in view of the teachings herein and may be accomplished without undue experimentation.

### The Active Agent

The active agent(s) described herein may comprise an agent, drug, compound, composition of matter or mixture thereof which provides some pharmacologic, often beneficial, effect. As used herein, the term further includes any physiologically or pharmacologically active substance that produces a localized or systemic effect in a patient. An active agent for incorporation in the pharmaceutical formulation described herein may be an inorganic or an organic compound, including, without limitation, drugs which act on: the peripheral nerves, adrenergic receptors, cholinergic receptors, the skeletal muscles, the cardiovascular system, smooth muscles, the blood circulatory system, synoptic sites, neuroeffector junctional sites, endocrine and hormone systems, the immunological system, the reproductive system, the skeletal system, autacoid systems, the alimentary and excretory systems, the histamine system, and the central nervous system. Suitable active agents may be selected from, for example, hypnotics and sedatives, tranquilizers, respiratory drugs, drugs for treating asthma and COPD, anticonvulsants, muscle relaxants, antiparkinson agents (dopamine antagonists), analgesics, anti-inflammatories, antianxiety drugs (anxiolytics), appetite suppressants, antimigraine agents, muscle contractants, anti-infectives (antibiotics, antivirals, antifungals, vaccines) antiarthritics, antimalarials, antiemetics, anepileptics, bronchodilators, cytokines, growth factors, anti-cancer agents, antithrombotic agents, antihypertensives, cardiovascular drugs, antiarrhythmics, antioxicants, anti-asthma agents, hormonal agents including contraceptives, sympathomimetics, diuretics, lipid regulating agents, antiandrogenic agents, antiparasitics, anticoagulants, neoplastics, antineoplastics, hypoglycemics, nutritional agents and supplements, growth supplements, antienteritis agents, vaccines, antibodies, diagnostic agents, and contrasting agents. The active agent, when administered by inhalation, may act locally or systemically. In some embodiments, the active agent may be a placebo.

The active agent may fall into one of a number of structural classes, including but not limited to small molecules, peptides, polypeptides, antibodies, antibody fragments, proteins, polysaccharides, steroids, proteins capable of eliciting physiological effects, nucleotides, oligonucleotides, polynucleotides, fats, electrolytes, and the like.

In embodiments of the invention, the active agent may include or comprise any active pharmaceutical ingredient that is useful for treating inflammatory or obstructive airways diseases, such as asthma and/or COPD. Suitable active ingredients include long acting beta 2 agonist, such as salmeterol, formoterol, indacaterol and salts thereof, muscarinic antagonists, such as tiotropium and glycopyrronium and salts thereof, and corticosteroids including budesonide, ciclesonide, fluticasone, mometasone and salts thereof. Suitable combinations include (formoterol fumarate and budesonide), (salmeterol xinafoate and fluticasone propionate), (salmeterol xinofoate and tiotropium bromide), (indacaterol maleate and glycopyrronium bromide), and (indacaterol and mometasone).

The amount of active agent in the pharmaceutical formulation will be that amount necessary to deliver a therapeutically effective amount of the active agent per unit dose to achieve the desired result. In practice, this will vary widely depending upon the particular agent, its activity, the severity of the condition to be treated, the patient population, dosing requirements, and the desired therapeutic effect. The composition will generally contain anywhere from about 1% by weight to about 100% by weight active agent, typically from about 2% to about 95% by weight active agent, and more typically from about 5% to 85% by weight active agent, and will also depend upon the relative amounts of additives contained in the composition. In embodiments of the invention compositions of the invention are particularly useful for active agents that are delivered in doses of from 0.001 mg/day to 10 g/day, such as from 0.01 mg/day to 1 g/day, or from 0.1 mg/day to 500 mg/day, or from 1 mg to 1 g/day. In embodiments of the invention compositions of the invention are useful for active agents delivered in doses in 10-1000 nanograms per day and/or per dose. It is to be understood that more than one active agent may be incorporated into the formulations described herein and that the use of the term “agent” in no way excludes the use of two or more such agents.

### Buffers/Optional Ingredients

Buffers are well known for pH control, both as a means to deliver a drug at a physiologically compatible pH (i.e., to improve tolerability), as well as to provide solution conditions favorable for chemical stability of a drug. In embodiments of formulations and processes of the present invention, the pH milieu of a drug can be controlled by co-formulating the drug and buffer together in the same particle.

Buffers or pH modifiers, such as histidine or phosphate, are commonly used in lyophilized or spray-dried formulations to control solution- and solid-state chemical degradation of proteins. Glycine may be used to control pH to solubilize proteins (such as insulin) in a spray-dried feedstock, to control pH to ensure room-temperature stability in the solid state, and to provide a powder at a near-neutral pH to help ensure tolerability. Preferred buffers include: histidine, glycine, acetate, citrate, phosphate and Tris.

Non-limiting optional excipients include salts (e.g., sodium chloride, calcium chloride, sodium citrate), antioxidants (e.g., methionine), excipients to reduce protein aggregation in solution (e.g., arginine), taste-masking agents, and agents designed to improve the absorption of macromolecules into the systemic circulation (e.g., fumaryl diketopiperazine).

Exemplary settings for a laboratory-scale spray dryer are as follows: an air inlet temperature between about 80° C. and about 160° C., such as between 100° C. and 140° C.; an air outlet between about 40° C. to about 100° C., such as about 50° C. and 80° C.; a liquid feed rate between about 1 g/min to about 20 g/min, such as about 3 g/min to 10 g/min; drying air flow of about 200 L/min to about 900 L/min, such as about 300 L/min to 700 L/min; and an atomization air flow rate between about 5 L/min and about 50 L/min, such as about 10 L/min to 30 L/min. The solids content in the spray-drying feedstock will typically be in the range from 0.5% w/v (5 mg/ml) to 10% w/v (100 mg/ml), such as 1.0% w/v to 5.0% w/v. The settings will, of course, vary depending on the scale and type of equipment used, and the nature of the solvent system employed. In any event, the use of these and similar methods allow formation of particles with diameters appropriate for aerosol deposition into the lung.

In some of the examples herein, process conditions used for generating the particles comprising the formulations are as follow; solids content of 0.5 to 4%; liquid feed rate of 2 to 5 mL per minute; drying gas flow rate of 200 to, 600 L per minute; atomizing gas flow rate of 20 to 30 L per minute; outlet temperature of 40 to 70° C. (and wherein an inlet temperature was set to generate the specified outlet temperature). Spray drying was done using a super Novartis Spray Dryer (sNSD), which is a custom-built lab-scale dryer. The sNSD has a volume capacity similar to that of a commercially-available lab scale spray dryer, such as the Bucchi B290 (Switzerland).

Particles made in accordance with embodiments of the process of the present invention may be formulated to be delivered in a variety of ways, such as orally, transdermally, subcutaneously, intradermally, intranasally, pulmonary, intraocularly, etc. In embodiments of the present invention, particles are prepared and engineered for inhalation delivery.

### Inhalation Delivery System

The present invention also provides a delivery system, comprising an inhaler and a dry powder formulation of the invention.

In one embodiment, the present invention is directed to a delivery system, comprising a dry powder inhaler and a dry powder formulation for inhalation that comprises spray-dried particles that contain a therapeutically active ingredient, wherein the in vitro total lung dose is between 50% and 100% w/w of the nominal dose, such as at least 55% or 60% or 65% or 70% or 75% or 80% or 85% of the nominal dose.

### Inhalers

Suitable dry powder inhaler (DPIs) include unit dose inhalers, where the dry powder is stored in a capsule or blister, and the patient loads one or more of the capsules or blisters into the device prior to use. Alternatively, multi-dose dry powder inhalers are contemplated where the dose is pre-packaged in foil-foil blisters, for example in a cartridge, strip or wheel. Formulations of the present invention are suitable for use with a broad range of devices, device resistances, and device flow rates. In embodiments of the invention, products and formulations of the present invention afford enhanced bioavailability.

The Novartis multidose blister inhaler (Aspire) as described in PCT Patent Application Publication WO 2017/125853 nominally comprises 30 doses contained in individual blisters each having a volume such that up to about 10 mg of conventionally engineered powder may be filled therein. The Aspire multi-dose powder inhalation device generally comprises a body comprising an interior cavity and a cartridge that is removably insertable into the interior cavity of the body, the cartridge comprising a mouthpiece through which aerosolized powder medicament may be delivered to a user, wherein the cartridge houses a strip of receptacles, each receptacle adapted to contain a dose of powder medicament, a piercing mechanism to open each blister and an aerosol engine.

Using Novartis Pulmosol or Pulmosphere engineered powders, with the Aspire multi-dose powder inhalation device, up to 50% drug loading can be achieved, resulting in a total drug delivery capacity of up to 150 mg. Such a delivery capacity exceeds by nearly a factor of 10 that of conventional multidose inhalers delivering conventional drug formulations. Using formulations and methods of the present invention having a product density of at least 50 with the Aspire multi-dose powder inhalation device, delivery efficiency is at least 2 to 3 times greater compared to that achievable with Pulmosol or Pulmosphere engineered powders, hence 2-3 times the amount of drug delivery capacity for the same size inhaler device. Moreover, this represents a potential 20 to 30 fold improvement over conventional inhalers with conventional drug formulations. Additionally, of course an inhaler could be made correspondingly smaller to yield the same 150 mg total drug delivery capacity.

A variety of receptacles may be used to contain the powders herein, most commonly, capsules and blisters. Blisters typically have a higher relative percentage of white space (void space) then do capsules, therefore, using conventional filling equipment, blisters cannot typically be filled to as high-capacity, proportionally, as can capsules. In some circumstances, this is simply a limitation of commercially available filling equipment. As a result, however, actual product densities of blisters may be slightly less than calculated product densities, and may further be smaller than those of capsules, or other receptacles which can be completely filled.

As described herein, it has been found that the novel metric of Compressibility Index can be a useful predictive tool to estimate the aerosolization of highly packed dense particles from the receptacles (see Equation 2). In embodiments of the invention, powders with a compressibility index of less than 20 have the best aerosol performance. Powders manufactured using fast drying conditions had a high compressibility index (greater than 20) and the ED of those powders were much lower than the powders made to have a low compressibility index. The compressibility index is derived from the tapped density (a measure of powder packing) and the puck density (a measure of powder compressibility). Embodiments of powders of the present invention comprising particles are designed to pack efficiently even at low forces applied to the powder, therefore, a large difference in packing density is not expected at higher forces. As a result, the formulation with increased packing density at higher applied force (using vacuum in this case) suggests that particles were physically interlocked and that these could not be easily aerosolized from the receptacles.

### Use in Therapy

Embodiments of the present invention provide a method for treatment of any disease or condition by which inhalation dosing is suitable. Embodiments of the invention are particularly suitable for inhalation delivery in drug/device combinations where it is desirable or beneficial to make the delivery devices smaller, and/or molecules requiring delivery of a high payload. As such, embodiments of the invention have applicability across a range of API potency. In particular, embodiments of the invention are useful with APIs which require higher and/or constant dosing, such as antibiotics and antibodies (or antibody fragments). Non-limiting examples include chemotherapeutics, hormones, inhaled proteins, siRNAs and other polynucleotides, and drug formulations having a high-excipient content (such as controlled release formulations). A further specific example of the utility of the formulations and methods of the present invention is the inhalation administration of powders for the treatment of infectious diseases.

Embodiments of the present invention provide a method for the treatment of respiratory, airway and lung diseases, for example obstructive or inflammatory airways disease, such as asthma and chronic obstructive pulmonary disease. The method comprises administering to a subject in need thereof an effective amount of a dry powder formulation made in accordance with embodiments herein.

Embodiments of the present invention provide a method for the treatment of systemic diseases, for example, infectious diseases, the method which comprises administering to a subject in need thereof an effective amount of the aforementioned dry powder formulation. Embodiments of compositions and methods of the present invention enable a therapeutic dose by single inhalation of the contents of a size 2 or smaller receptacle.

### COMPARATIVE EXAMPLES—STATE-OF-THE-ART FOR IMPROVING DOSING

### Comparative Example 1

The Novartis Podhaler® device is a unit dose, capsule-based dry powder inhaler of low-medium resistance (R=0.08 cmH₂O^{1/2} L⁻¹ min). A TOBI® Podhaler® therapeutic dose consists of inhaling the contents of four size 2 hypromellose capsules, each containing about 50 mg of spray-dried PulmoSphere™ powder (about 200 mg powder/therapeutic dose). The drug substance, tobramycin sulfate, comprises about 85% w/w of the powder composition (i.e., about 170 mg tobramycin sulfate/therapeutic dose, or 112 mg as tobramycin/therapeutic dose). In vitro studies reveal that about 60% of the powder mass is delivered to the lungs of CF patients (i.e., about 100 mg tobramycin sulfate).

An administration time for the dry powder formulations is about 1 minute for drug products requiring inhalation of powder from a single capsule, and on the order of 5 to 6 minutes for tobramycin inhalation powder (4 capsules). A clear advantage for dry powders is that, other than simply wiping the mouthpiece, the devices do not require cleaning and disinfection. This dramatically reduces the daily treatment burden to between 2 and 12 minutes for the products discussed above. However, the need to administer four discrete capsules in TOBI Podhaler increases the potential for patient errors associated with capsule handling and dose preparation. Hence, it is advantageous to fill and administer the entire nominal dose in a single receptacle, if possible.

### Comparative Example 2

Colobreathe contains 125 mg of neat micronized colistimethate in a size 2 capsule. It is administered over three or more inhalations with the Turbospin® (PH&T, Milan, Italy) device. The Colbreathe drug device combination can be considered to represent the highest drug payload in a commercially available device. However, the total dose delivered in a single capsule affords a low TLD which results in a low product density (see Table 4). As a result, at least three inhalations are required to administer a therapeutic dose.

### EXPERIMENTAL DATA—EXAMPLES ACCORDING TO EMBODIMENTS OF THE PRESENT INVENTION

### Example 1. Spray-Dried Powders Comprising an Antibody Fragment

Spray dried powder formulations comprising an antibody fragment (CSJ-117), were prepared using the sNSD spray dryer. The formulations contained 50% w/w CSJ-117, 0-15% w/w of trileucine (as shell former), 25-35% w/w saccharide and 3-10% w/w buffering agents. Some samples were spray dried under fast drying conditions in order to generate low density particles. Spray dryer parameters consistent with fast drying conditions comprise the solids content of 1 to 2%, a liquid feed rate of 5 to 10 mL per minute; drying gas flow rate of 500 to 600 L per minute; atomizing gas flow rate of 20 to 30 L per minute and an outlet temperature of 60 to 70° C. (and wherein an inlet temperature was set to generate the specified outlet temperature). Other samples were spray dried under slow drying conditions to generate denser particles. Spray-dryer parameters consistent with slow drying conditions comprise a solids content of 1-I have no 3.5%, a liquid feed rate of 2.5 to 5 mL per minute; drying gas flow rate of 200 to 400 L per minute; atomizing gas flow rate of 20 to 30 L per minute and an outlet temperature of 50 to 55° C. (and wherein an inlet temperature was set to generate the specified outlet temperature).

Spray-dried powders of an antibody fragment (CSJ117) with no shell-forming excipient are characterized by spherical particles with a smooth particle morphology, a specific surface area (SSA). SSA is a property of solids defined as the total surface area of a material per unit of mass. Typically, larger surface area is achieved if particles are poor bit corrugated or porous. SSA as reported herein was measured using the Brunauer-Emmett-Teller (BET) analysis method. An aliquot of powder (about 500 mg) was added to a 1 mL volume sample tube and degassed for 960 minutes at 25° C. prior to analysis. Nitrogen was the analysis absorptive, and was analysis was conducted on a Micromeritics Tri-Star II Surface Area and Porosity Analyzer running MicroActive software. The BET SSA of particles with no shell-former was 6.35 m²/g with a tapped density of 0.32 g/ml (See Table 2, sample 7). Introduction of 15% w/w of the shell-forming excipient trileucine into the formulation (sample 10) results in a corrugated particle morphology, and increases the SSA to 11.8 m²/g. This yields a tapped density of 0.31 g/ml, which is surprisingly similar to that of sample 7. It is thought that decreases in particle density achieved with the increase in particle corrugations are offset by decreases in void spaces between particles resulting from lower interparticle cohesive forces. Powders of even higher SSA exhibit lower tapped densities, as the impact of decreased particle density outweighs improvements in particle packing. It has been surprisingly discovered that addition of small amounts of trileucine (2.5%) results in dramatic increases in tapped density to about 0.60 g/ml (FIG. 8 and Table 2, samples 1 and 2 This finding is unexpected, because prior art spray dried engineered particles exhibit high compressibility (greater than about 20) because they are corrugated and possess low density. The prior art engineered particles were designed to possess low particle density. In contrast, the present invention is designed to increase both powder density and packing density in order to fill more powder mass into a given size receptacle. If the particles are not properly designed in accordance with embodiments of the present invention, the powders become very cohesive (gravitationally unstable) and do not yield desirable packing an aerosol properties. Hence, powders, according to embodiments of the present invention result in particles which are denser, and less corrugated, yet maintain good aerosol properties, including dispersibility. This occurs despite no significant increases in the SSA of the particles relative to particles without shell-forming excipient (FIG. 7A).

It is believed that the small amount of shell-forming agent reduces interparticle cohesive forces, enabling closer packing of particles in the bulk powder, in spite of their low Pe. All the lots prepared at slow drying conditions showed higher tapped densities as compared to those prepared at fast drying conditions (Table 2. The amount of shell-former added to the formulations is carefully controlled because further increases in trileucine content decrease the tapped density due to increased surface corrugation of the particles. This is, of course, undesirable for the goal of high product-density formulations.

Additional trends are apparent in the data reported in Table 2. Particles with low trileucine contents and slow drying rates exhibit higher tapped densities, lower compressibility indices, higher emitted doses, and a much more consistent emptying pattern with a portable dry powder inhaler for high fill masses (See FIG. 5). Thus samples 1-6 were made with a process comprising slow drying rates (low Pe), and samples 7-12 were made with a process comprising fast drying rates (high Pe). The mean ED for a powder with a low Pe (sample 4) was 86% compared to 58% for powder (sample 8) with a high Pe.

TABLE 2
Physical characteristics of spray-dried powders comprising an antibody fragment. Samples 1-6
were powders made under slow drying conditions (low Pe) with a dryer outlet temperature of
55° C., and dry air flow at 300 L/min. Samples 7-12 were powders made under fast drying
conditions (high Pe) with a dryer outlet temperature of 70° C., and dry air flow of 600 L/min.

| | | Trileu | Solids | SSA | ρ_{tapped} | ρ_{puck} | Drying | Compressibility | Emitted Dose |
|---|---|---|---|---|---|---|---|---|---|
| Sample | Lot | (%) | (%) | (m²/g) | (g/ml) | (g/ml) | Conditions | Index | (% w/w) (RSD)* |
| 1 | 761-72-02 | 2.5 | 1.0 | 6.01 | 0.57 | 0.64 | slow | 10.9 | 83 (3) |
| 2 | 761-72-04 | 2.5 | 2.5 | 6.30 | 0.57 | 0.65 | slow | 12.3 | 86 (3) |
| 3 | 761-72-07 | 2.5 | 3.5 | 6.35 | 0.45 | 0.53 | slow | 15.1 | 85 (1) |
| 4 | 761-58-07 | 5 | 1.0 | 6.65 | 0.59 | 0.58 | slow | 1.72 | 86 (3) |
| 5 | 761-72-05 | 5 | 2.5 | 6.82 | 0.51 | 0.58 | slow | 12.1 | 91 (1) |
| 6 | 761-72-03 | 15 | 1.0 | 9.81 | 0.34 | 0.44 | slow | 22.7 | n/a^{§} |
| 7 | 761-58-10 | 0 | 1.0 | 6.35 | 0.32 | 0.38 | fast | 15.8 | n/a^{§} |
| 8 | 761-32-02 | 5 | 1.0 | 8.33 | 0.45 | 0.58 | fast | 22.4 | 58 (23) |
| 9 | 761-72-01 | 10 | 1.0 | 9.50 | 0.44 | 0.49 | fast | 10.2 | 72 (2) |
| 10 | 761-32-01 | 15 | 1.0 | 11.8 | 0.31 | 0.42 | fast | 45.2 | n/a^{§} |
| 11 | 761-22-02 | 15 | 1.0 | 12.7 | 0.15 | 0.38 | fast | 60.5 | n/a^{§} |
| 12 | 761-22-03 | 15 | 1.0 | 14.9 | 0.14 | 0.28 | fast | 50.0 | n/a^{§} |

In Table 2 above, it can be seen that samples 1-5 are desirably dense, with a low Compressibility Index and correspondingly high emitted dose. Samples 1-5 contain trileucine in amounts between 2.5 and 5%. Sample 6 contains 15% trileucine, and is insufficiently dense, with a comparatively high Compressibility Index (greater than 20). Sample 8, dried under fast drying conditions also has a high compressibility index. Samples 9-12 also contain high levels (10-15%) of trileucine, and have corresponding high Compressibility Indices, except for sample 9 which has a low Compressibility Index. Emitted doses for samples 6-7 and 10-12 were not measured because the target 150 mg fill mass was not achieved.

Emitted dose delivery performance for the Table 2 samples was tested using the Novartis Podhaler dry powder inhaler. A target of 150 mg of each powder formulation was filled (or attempted to be filled) into a size 2 HPMC capsule. The 150 mg target fill is 80% of the fill volume for the powder at a puck density of 0.5 mg/mL. The was discharged into a customized dose uniformity sampling apparatus (DUSA) at an air flow rate of 90 L per minute for 1.3 seconds to draw 2 L of air, producing a pressure drop across the device of approximately 2 kPa. The ED values reported are a mean of three replicates and expressed as a percentage of fill mass.

### Example 2. Spray-Dried Powders Comprising a Small Molecules

Spray-dried formulations of two antibiotics, levoffloxacin and gentamycin sulfate, and a β2-adrenergic agonist, albuterol sulfate, were prepared using a lab scale spray dryer (a custom design super Novartis Spray Dryer, sNSD).

TABLE 3
Formulation details and the Physical characteristics of spray-dried powders comprising
antibiotics and a a β2-adrenergic agonist. Samples were made under slow drying conditions
(low Pe) with a dryer outlet temperature of 50-55° C., and dry air flow at 300 L/min.

| | Drug | | | | | | Emitted Dose |
|---|---|---|---|---|---|---|---|
| | loading | Formulation | Solids | ρ_{tapped} | ρ_{puck} | Compressibility | (% w/w) |
| Sample | (%) | (%) | (%) | (g/ml) | (g/ml) | Index | (RSD) |
| Levofloxacin | 80 | Trileucine (5.0) | 1.0 | 0.54 | 0.61 | 11.5 | 64 (6) |
| | | Mannitol (12.0) | | | | | |
| | | Buffering agents | | | | | |
| | | (3%) | | | | | |
| Gentamycin | 30 | Trileucine (5.0) | 1.0 | 0.56 | 0.63 | 11.1 | 79 (5) |
| Sulfate | | Trehalose (62.0) | | | | | |
| | | Buffering agents | | | | | |
| | | (3%) | | | | | |
| Albuterol | 30 | Trileucine (5.0) | 1.0 | 0.67 | 0.68 | 1.5 | 84 (2) |
| Sulfate | | Trehalose (62.0) | | | | | |
| | | Buffering agents | | | | | |
| | | (3%) | | | | | |

Emitted dose delivery performance for samples in Table 3 was tested using the Novartis Podhaler dry powder inhaler. A target of 150 mg of each powder formulation was filled (or attempted to be filled) into a size 2 HPMC capsule. The 150 mg target fill is 80% of the fill volume for the powder at a puck density of 0.5 mg/mL. The was discharged into a customized dose uniformity sampling apparatus (DUSA) at an air flow rate of 90 L per minute for 1.3 seconds to draw 2 L of air, producing a pressure drop across the device of approximately 2 kPa. The ED values reported are a mean of three replicates and expressed as a percentage of fill mass.

### Comparative Example 3. Calculated Product Densities for Various High-Dose Drug-Device Combinations

Product density data for several currently-marketed high-dose formulations (as defined by TLD greater than 10 mg), are shown in Table 4 below, as is data for a third-party proprietary salt formulation technology, which utilizes dense salts to increase particle density to enable high dose delivery. Currently-marketed high dose formulations with a TLD greater than 10 mg include TOBI Podhaler (Novartis) and Colobreathe (Forest). These products have a product density, as determined by the methods herein, of about 30 to 50 mg/ml. The product density of the levofloxacin formulated using the proprietary salt formulation technology was calculated based on publicly reported data.

Table 4 below shows calculated product densities of various conventional and/or prior art drug/device combinations.

TABLE 4

| | | Receptacle | | | Product |
|---|---|---|---|---|---|
| | API dose | volume | TLD | TLD | density |
| Product | mg | mL | mg | % w/w | mg/mL |
| TOBI Podhaler | 28 | 0.37 | 17.6 | 63 | 47.6 |
| Exubera | 3 | 0.05 | 1.2 | 40 | 24 |
| Colobreathe | 125 | 0.37 | 14.0 | 11 | 37.8 |
| Proprietary salt | 34 | 0.27 | 8.5 | 25 | 31.5 |
| formulation of | | | | | |
| levofloxacin | | | | | |
| Nedrocromil | 4.2 | 0.37 | 0.56 | 13 | 1.5 |
| Spinhaler | | | | | |
| Advair Diskus | 0.25 | 0.03 | 0.05 | 20 | 1.2 |
| (FP) | | | | | |
| Spiriva | 0.018 | 0.27 | 0.27 | 20 | 0.013 |
| Handihaler | | | | | |
| OnBrez | 0.34 | 0.27 | 0.05 | 15 | 0.19 |
| Breezhaler | | | | | |
| Ventolin | 0.2 | 0.37 | 0.014 | 7 | 0.038 |
| Rotahaler | | | | | |
| Foradil Aerolizer | 0.012 | 0.27 | 0.0024 | 20 | 0.009 |
| Bronchitol | 40 | 0.27 | 12 | 40 | 44 |

In contrast to the product densities of these marketed products, the spray-dried formulations of the present invention have much greater product densities, between 150 and 250 mg/ml. These values were achieved while filling a single receptacle (0.095 mL) without any dose compression, or mechanical means to increase filling density. That is, the increase in product density is achieved entirely by the inventive formulation according to embodiments of the present invention.

An important aspect of the powders of the present invention is that of a high TLD—about 60% w/w or greater—when expressed as a percentage of the nominal dose. It is clear that the presence of the shell-forming excipient and the smaller geometric size of low Pe formulations enables high delivery efficiencies to be achieved with portable dry powder inhalers (e.g., the Novartis Podhaler® or Breezhaler™ DPIs).

Table 5 below shows calculated product densities of formulations made according to embodiments of the present invention. Each of the formulations in Table 5 are made in accordance with Example 1 or 2 of the present invention, and comprise the active as indicated.

TABLE 5

| | API | Receptacle | | | Product | |
|---|---|---|---|---|---|---|
| | dose | volume | TLD | TLD | density | |
| Active | mg | mL | mg | % w/w | mg/mL | Example |
| Antibody | 75 | 0.37 | 54.9 | 74 | 148.4 | 1 |
| fragment | | | | | | |
| Levofloxacin | 120 | 0.37 | 82.5 | 69 | 223.8 | 2 |
| Gentamicin | 45 | 0.37 | 33.4 | 74 | 90.2 | 2 |
| Albuterol | 45 | 0.37 | 33.4 | 74 | 90.2 | 2 |
| Sulfate | | | | | | |

As demonstrated by Table 5, product density can be increased up to 223 mg/mL or higher, by increasing the drug loading to 80-90%. The APIs and percentage drug loading shown in Table 5 are randomly selected and presented to show that performance of formulations of the present invention is not impacted by the choice of API or by the drug loading.

In addition to high-payload delivery in single-use or unit-dose devices, the powders of this invention provide significant advantages in multi-dose devices (MD-DPIs). A key design constraint in such devices is portability, as dictated by the overall size of the device. This, in turn, governs the number of possible doses and limits the size of the individual dosing receptacles (e.g., blister cavity size). The powders of the present invention have product densities which enable fill masses on the order of 10 mg and TLD of about 7 mg in a multi-dose dry powder inhaler with a receptacle volume of just 0.1 ml. This potentially enables new classes of drugs to be introduced into MD-DPIs.

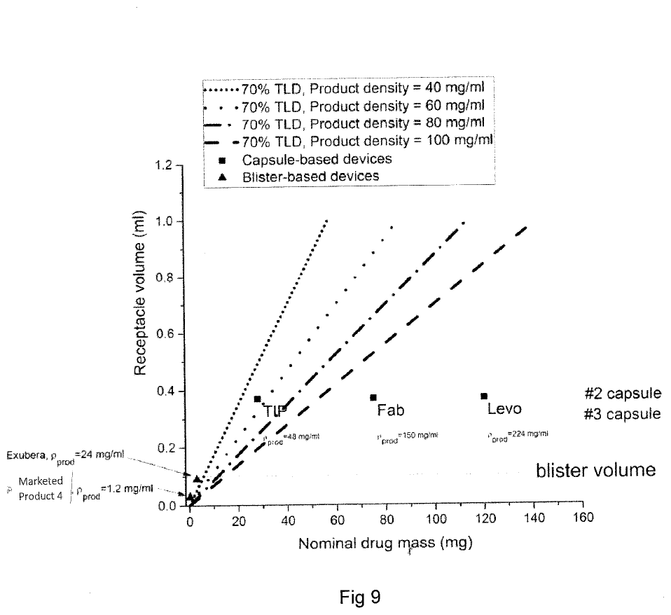

FIG. 9 is a plot of nominal drug mass versus receptacle volume at 70% TLD for four different product densities of embodiments of the present invention. Three conventional product density points are additionally plotted on this graph: (i) Novartis' tobramycin inhalation powder (labeled “TIP”); (ii) a formulation comprising an antibody fragment (labeled “FAB”); and (iii) a formulation comprising levofloxacin (labeled “Levo”). A first dotted line parallel to the X axis represents a hypothetical blister receptacle for a small, portable multidose blister based inhaler, at a 0.1 mL volume capacity. Two parallel dotted lines respectively represent the volumes of number two and three sized capsules. Based upon these data, a typical drug mass would need to be approximately 8 mg at the 60% product density, approximately 11 mg at the 80% product density and approximately 40 mg at the hundred milligrams per milliliter product density.

Alternatively, these powders can be introduced into unit-dose or single-dose disposable DPIs. In capsule-based inhalers with a size 2 capsule, TLD on the order of 100 mg can be achieved. For a size 0 capsule, TLD on the order of 200 mg can be achieved. This enables the lowest potency drugs (e.g., anti-infectives) to be effectively delivered by inhaling the contents from a single receptacle. For a size 2 capsule, most subjects can empty the contents of the capsule in a single inhalation, provided they can achieve an inhaled volume of at least about 1.2 L.

Having now fully described this invention, it will be understood to those of ordinary skill in the art that the methods and formulations of the present invention can be carried out with a wide and equivalent range of conditions, formulations, and other parameters without departing from the scope of the invention or any embodiments thereof.

All patents and publications cited herein are hereby fully incorporated by reference in their entirety. The citation of any publication is for its disclosure prior to the filing date and should not be construed as an admission that such publication is prior art.

1. A spray-dried pharmaceutical powder composition comprising particles deliverable from a dry powder inhaler, the composition comprising active agent, and about 0.5 to 10% by weight of a shell-forming excipient, wherein the powder is characterized by a product density greater than 50 mg/ml.

2. (canceled)

3. The composition and of claim 1 wherein the receptacle comprises a capsule having a volume capacity of 0.21 to 0.5 mL, and wherein the powder is characterized by a product density greater than 80 mg/mL.

4. The composition of claim 1 where the shell-forming excipient comprises leucine or trileucine.

5. The composition of claim 1, wherein the particles are characterized by rugosity of 1-3.

6. The composition of claim 1 wherein the product density is greater than 100 mg/mL.

7. The composition of claim 1 where the powder is spray dried under process conditions characterized by a Peclet number of 0.5 to 3.

8. The composition of claim 1 wherein the shell forming excipient is present in an amount such that the specific surface area of the particles in the presence of the shell-forming excipient is comparable to like-sized particles comprising no shell-former.

9. The composition of claim 1 wherein the powder is characterized by a compressibility index of less than 20.

10. The spray-dried pharmaceutical powder composition of claim 1 wherein the powder is made by a process comprising:
preparing a feedstock comprising a solvent, active agent and 0.5-10% of a shell forming excipient;
spray drying the feedstock under process conditions characterized by a Peclet number of 0.5 to 3; and,
collecting the resulting powder, wherein the powder is characterized by a product density greater than 50 mg/mL, and a compressibility index of less than 20.

11. (canceled)

12. (canceled)

13. The powder of claim 10 wherein the shell-forming excipient is present in an amount such that the specific surface area of particles in the presence of the shell forming excipient is comparable to like sized particles comprising no shell-forming excipient.

14. A method of delivering a plurality of particles comprising a therapeutic dose of an active pharmaceutical agent to the lungs of a subject, the method comprising:
a. preparing a solution of an active agent and shell-forming excipient in a solvent wherein the shell forming excipient is present between 2 and 5%,
b. spray drying the solution to obtain a powder comprising particulates, wherein the powder is characterized by a product density of at least about 80 mg/mL
c. packaging the spray-dried powder in a receptacle; and
d. providing an inhaler having a means for extracting the powder from the receptacle, wherein the powder, when administered by inhalation, provides at least 70% lung deposition.

15. A method for the treatment of a disease or condition which comprises administering to a subject in need thereof an effective amount of a dry powder formulation include according to claim 1.

16. The method of claim 15 wherein the disease or condition comprises an obstructive or inflammatory airways disease.

17. The method of claim 15 wherein the disease or condition comprises an infectious disease, and wherein a therapeutic dose is delivered in a single size 2 or smaller receptacle.

18. A delivery system comprising an inhaler and a dry powder formulation according to claim 1.

19. The delivery system of claim 18 wherein the inhaler comprises a blister-based multidose inhaler

20. The composition of claim 1 wherein the active agent comprises an antibiotic.

21. A multiple dose powder inhalation device and drug combination comprising:
a body comprising an interior cavity and a cartridge that is removably insertable into the interior cavity of the body, the cartridge comprising a mouthpiece through which aerosolized powder medicament may be delivered to a user, wherein the cartridge houses a strip of receptacles, each receptacle adapted to contain a dose of powder medicament, a piercing mechanism to open each blister and an aerosol engine, and
a powder medicament contained within each receptacle, wherein the powder medicament comprises a spray-dried pharmaceutical powder composition comprising particles deliverable from a dry powder inhaler, the composition comprising active agent, and about 0.5 to 10% by weight of a shell-forming excipient, wherein the powder is characterized by a product density greater than 50 mg/ml.

22. The inhalation device and drug combination of claim 21 wherein a total drug delivery capacity is greater than about 300 mg.

23. (canceled)

24. The inhalation device and drug combination of claim 21 wherein receptacle is a blister, having a volume less than 0.95 mL, and a total lung dose of at least 5 mg can be delivered from the blister.
